(19) United States
(12) Patent Application Publication
(10) Pub. No.: US 2024/0189538 A1
(43) Pub. Date: Jun. 13, 2024

(54) Methods and Compositions for Noninvasive Delivery of Acoustic Frequencies

(71) Applicant: Linda Louise Bamber, Independence, MO (US)

(72) Inventor: Linda Louise Bamber, Independence, MO (US)

(21) Appl. No.: 16/952,027

(22) Filed: Nov. 18, 2020

Related U.S. Application Data
(60) Provisional application No. 62/936,901, filed on Nov. 18, 2019.

(51) Int. Cl. A61M 21/00; A61H 23/02

(52) U.S. Cl. CPC A61M 21/00; A61H 23/0236; A61H 2201/0157; A61H 2205/04; A61H 2205/088; A61H 2205/12; A61M 2021/0027; A61M 2210/083

(57) ABSTRACT

The present invention provides methods, devices, and systems for the noninvasive delivery of one or more oscillations that comprise multiple auditory frequencies to a user's cells or organ systems. The combinations of multiple auditory frequencies that are used in the methods, devices, and systems have been identified as, or are believed anecdotally, to be therapeutic. Advantageously, devices of the invention require only small amounts of energy so they can be configured in a variety of ways so that the user can remain mobile. Further, the minimal amount of energy required to operate such devices allows for greater variation in the combinations of frequencies that comprise the oscillations relative to comparable devices.

### BACKGROUND OF THE INVENTION

### A. Field of the Invention

The present invention relates to methods, systems, and compositions to transmit low frequency sounds to a body through noninvasive means.

### B. Description of the Related Art

For thousands of years, humans have used music and various sounds to evoke emotions, inspire, or reduce stress among other objectives. More recently, audible sounds or electrical stimulations have been used to treat stress or various psychological diseases or conditions by using noninvasive delivery methods. Such noninvasive delivery methods include headphones or similar means of delivery to the auditory canals of a person or electrical pulses through a chair, pads, or other means through which electrical impulses can be sent. These methods have shown promise for alleviating stress or certain psychological diseases. The equipment for performing these methods is often immobile and expensive.

More recently, mobile, delivery devices have been designed to deliver small electrical or electromagnetic impulses to treat mental health or relieve pain. These devices appear to be primarily designed to be implanted or attached to the body at or near the site to be treated. For example, to stimulate an area of the brain a device, e.g. headphones or earbuds, is attached to the head and treatment is either delivered through the auditory canals or, alternatively, through implanted electrodes. To stimulate the vagus nerve, a device is often implanted in the chest to deliver electrical impulses. While these devices have been shown to have therapeutic effects, they either require a surgical procedure or restrict a user's movements and abilities during treatment. For example, during treatment through the auditory canals, the user cannot hear normally and their communications are generally restricted to non-sound producing means.

Attempts have been made to treat other conditions, such as tremors, using some form of transcutaneous peripheral nerve stimulation. Challenges associated with these attempts include the physical variations in body sizes and dimensions among individuals, as well as the amount of power necessary to deliver therapeutic electrical or electromagnetic impulses for a given stimulation session. For instance, in a wrist-worn device targeting the median, ulnar, and radial nerves at the wrist, the amount of power needed for a given stimulation session can vary widely based on skin impedance and usage scenarios. Because of these challenges, these devices only deliver a restricted number of energy frequencies. To date, these impediments have not been fully overcome.

### SUMMARY OF THE INVENTION

The present invention provides methods, devices, and systems relating to the noninvasive delivery of acoustic frequencies to a body that overcome deficiencies in the art. Advantageously, the compositions and methods of the invention provide a wide variety of combinations of different acoustic frequencies through noninvasive devices of the invention that require relatively small amounts of energy to operate and allow the user to remain mobile.

The present invention provides a method of treating a user that comprises positioning a device adjacent to an outer skin surface of the user; and emitting an oscillation from the device near an outer skin surface of the user such that at least one cell within the user receives the oscillation. The oscillation comprises a combination of multiple acoustical frequencies that are emitted together for a pre-determined amount of time. Oscillations are about 4 to 30 minutes in length. An exemplary preferred length is 8 minutes.

Alternatively, an oscillation may comprise a combination of multiple acoustical frequencies that are emitted separately or one at a time. In such cases, the frequencies may be emitted in a preferred order or a random order.

The present invention also provides a method of treating a user that comprises emitting a program from a device. Herein, a program (or set) is a combination of two or more oscillations (or songs). The oscillations comprising a program are played sequentially. For example, a user may be treated with a program that comprises first part that is a detoxification song, a second part that is an inflammation song, and then a third part that is a stress relieving song. A program or set may comprise more than three oscillations or songs. The number and order of the oscillations that comprise a program depends upon the intent of the user. Herein, programs are longer than 30 minutes in length.

In preferred embodiments, an oscillation (song) comprises multiple frequencies that are played or transmitted together at one time. Less preferably, the multiple frequencies of an oscillation can be played separately and either in a preferred order or randomly.

Preferably, one oscillation comprises 1-30 frequencies. Also preferably, one oscillation (or song) is 4 to 30 minutes in length. Usually, oscillations are 8 minutes in length. Alternatively, oscillations may be as short as 30 seconds, 1, 2, or 3 minutes in length.

A program (or set) is a combination of multiple oscillations (or songs) that are played in a specified sequence or order. Programs are designed to treat a particular complaint identified by a user. Thus, the treatment may include multiple steps and each step is associated with a specific oscillation or song. For example, a user may wish to seek treatment of a benign cyst. The user selects the desired program. Unbeknownst to the user, the program comprises multiple oscillations. A first oscillation may be a detoxification song; a second oscillation may be an anti-inflammation song; and a third oscillation may be a stress-relieving song. Most commonly, programs are longer than 30 minutes because they include combinations of 2 or more oscillations.

Preferably, the acoustical frequencies that are used in the invention are within the range of 1 to 20,000 Hz. Also preferably, acoustical frequencies are emitted together for several seconds to a few minutes. Alternatively, but less desirably, acoustical frequencies are emitted separately and successively.

Advantageously, methods of the invention provide that the oscillation is transmitted through the outer skin surface to the user's vagus nerve in a noninvasive manner.

It is envisioned that methods, devices, and systems of the invention are useful for treating pain, inflammation, acne, allergies, arthritis, ankylosing spondylitis, asthma, bursitis, neuropathy, carpel tunnel syndrome, colitis, constipation, infection, diarrhea, dizziness, Meniere's disease, tinnitus, dystonia, fibromyalgia, kidney calculi, cholecystitis, headaches, migraines, hemorrhoids, hernias, irritable bowel syndromes, laryngitis, lumbago, cramps, numbness, tremors, peripheral nervous system disease, pulmonary fibrosis, Raynaud's disease, sinusitis, Tourette's syndrome, trigeminal neuralgia, urinary tract infections, bladder disease or disorders, varicose veins and venous insufficiency, breast diseases, alopecia, amenorrhea, cysts, mastitis, ademona, dysmenorrhea, endometriosis, respiratory disease or infection, or a stress or psychological disorder.

The invention provides that the oscillation that is emitted at or near an outer skin surface stimulates a vibrational energy to the outer skin surface and the area beneath the outer skin surface. Preferably, the invention also provides devices that are wearable and configured to be worn around a user's wrist, ankle, neck, or waist. Alternatively, devices of the invention can be attached to clothing, such as an undershirt, in close proximity to the skin and capable of emitting an oscillation that the oscillation is transmitted through the outer skin surface to the user's cells, such as the cells of the vagus nerve, in a noninvasive manner.

Devices of the invention include having a means of being worn by or attached to a user such that the device is in direct contact with or adjacent to the user's skin; and a means of emitting an oscillation such that the oscillation is emitted towards the user's skin such that at least one cell within the user receives the oscillation, and the oscillation comprises one or more combinations of multiple acoustical frequencies that are emitted together for a pre-determined amount of time.

Advantageously, devices of the invention require only small amounts of energy so they can be configured in a variety of ways so that the user can remain mobile. Further, the minimal amount of energy required to operate such devices allows for greater variation in the combinations of frequencies that comprise the oscillations relative to comparable devices.

Devices of the invention are capable of emitting acoustical frequencies within the range of 1 to 20,000 Hz. Such devices are capable of emitting each acoustical frequency is emitted for several seconds to a few minutes.

The invention also provides systems for treating a user that comprise a device having a means of being worn by or attached to a user such that the device is in direct contact with or adjacent to the user's skin; and a means of emitting an oscillation such that the oscillation is emitted towards the user's skin such that at least one cell within the user receives the oscillation, and the oscillation comprises one or more combinations of multiple acoustical frequencies that are emitted together for a pre-determined amount of time; and using the device to treat the user for pain, inflammation, acne, allergies, arthritis, ankylosing spondylitis, asthma, bursitis, neuropathy, carpel tunnel syndrome, colitis, constipation, infection, diarrhea, dizziness, Meniere's disease, tinnitus, dystonia, fibromyalgia, kidney calculi, cholecystitis, headaches, migraines, hemorrhoids, hernias, irritable bowel syndromes, laryngitis, lumbago, cramps, numbness, tremors, peripheral nervous system disease, pulmonary fibrosis, Raynaud's disease, sinusitis, Tourette's syndrome, trigeminal neuralgia, urinary tract infections, bladder disease or disorders, varicose veins and venous insufficiency, breast diseases, alopecia, amenorrhea, cysts, mastitis, ademona, dysmenorrhea, endometriosis, respiratory disease or infection, or a stress or psychological disorder.

In systems of the invention, the oscillation comprises a combination of multiple acoustical frequencies that are emitted together for a pre-determined amount of time. Preferably, the acoustical frequencies are within the range of 1 to 20,000 Hz, and each acoustical frequency is emitted for several seconds to a few minutes.

It is preferred that in systems of the invention, the oscillation is transmitted through the outer skin surface to an interior cell of the user. The interior cell may be in any targeted organ or system of the user.

Systems of the invention also provide that the emitting of the oscillation near a user's outer skin surface stimulates a vibrational energy to the outer skin surface and area beneath the outer skin surface.

Herein, a user is either a human or a non-human animal. Examples of non-human animals include cats, dogs, horses, goats, sheep, birds, or another species of animals that can benefit from use of the invention and can wear a device of the invention adjacent to or in contact with their skin. In the instances where the user is a non-human, a human operates the inventive device and selects the program or oscillation to be used.

Other features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description. Further, while specific advantages of the invention are detailed herein, various embodiments may include some, none, or all of these enumerated advantages.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description presented herein. Unless specifically noted, articles depicted in the drawings are not necessarily drawn to scale.

FIG. 1. An exemplary embodiment of the invention in the form of a watch.

FIG. 2. The embodiment of FIG. 1 in a partially open position.

FIG. 3. The embodiment of FIG. 1 in a fully open position.

FIG. 4. Frontal view of the embodiment of FIG. 1. in operational or turned-on mode.

FIG. 5. A lateral view of the embodiment of FIG. 1.

FIG. 6. A lateral view of the embodiment of FIG. 1 in a partially open position.

FIG. 7. A lateral view of the embodiment of FIG. 1 in a fully open position.

FIG. 8. Exploded view of the embodiment of FIG. 1.

FIG. 9. Another exemplary embodiment of the invention.

### DETAILED DESCRIPTION

The invention utilizes resonant frequencies to create precise oscillations. It is proposed that these oscillations can penetrate the skin in a noninvasive manner to generate a response in the targeted cells of the body. It is proposed that just electrical impulses can move from an initial cell to an adjacent cell and then to other cells, vibrations from sound can move from one cell to another to reach a target, e.g. the parasympathetic nervous system, vagus nerve, or other target.

The present invention provides as many as 750 different combinations of acoustical frequencies for various applications. It is proposed that these different combinations of frequencies can be used for the following types of applications: the treatment of pain, inflammation, acne, allergies, arthritis, ankylosing spondylitis, asthma, bursitis, neuropathy, carpel tunnel syndrome, colitis, constipation, infection, diarrhea, dizziness, Meniere's disease, tinnitus, dystonia, fibromyalgia, kidney calculi, cholecystitis, headaches, migraines, hemorrhoids, hernias, irritable bowel syndromes, laryngitis, lumbago, cramps, numbness, tremors, peripheral nervous system disease, pulmonary fibrosis, Raynaud's disease, sinusitis, Tourette's syndrome, trigeminal neuralgia, urinary tract infections, bladder disease or disorders, varicose veins and venous insufficiency, breast diseases, alopecia, amenorrhea, cysts, mastitis, ademona, dysmenorrhea, endometriosis, and respiratory disease and infection, and stress related disorders.

Each combination can include as many as 30 different acoustic frequencies. In some combinations, certain frequencies can occur more than once. In such combinations, it is preferred that the same frequency is not repeated in consecutive order. Rather, it is preferred that one or more different frequencies intervene between the occurrences of a repeated frequency. Preferred combinations are 4 minutes to 30 minutes in length.

The combinations below are exemplary of those that can be used in devices, systems, and methods of the invention.

An exemplary combination of acoustic frequencies that are anecdotally reported to be effective against colds and similar infections is emitted in the following order 20, 120, 146, 440, 444, 465, 727,776, 787, 880, 1500, 1550, 5000, 1000, 652, 725, 746, 751, 768, 110, 333, 666, 542, 522, 5500, 4400, 802, 720, 552, and 800 Hz.

An exemplary combination of acoustic frequencies that are anecdotally reported to be effective for intestinal detoxification is emitted in the following order 300, 320, 340, 360, 72, 95, 125, 165, 200, 333, 428, 444, 465, 522, 555, 600, 625, 650, 666, 690, 727, 787, 802, 880, 1250, 1500, and 1865 Hz.

An exemplary combination of acoustic frequencies that are anecdotally reported to be effective for immunogenic support is emitted in the following order 353, 320.007, 425.89, 459.12, 430.6, 254, 300, 433.13, 443.52, 2100, 11680, 14750, 11875, 15420, 17032.5, 5009.39, 9980.75, 375, 9000, 11090, 10000, 14375, 9375, 13340, 625, 12906.25, 470, 520, and 620 Hz.

An exemplary combination of acoustic frequencies that are anecdotally reported to be effective for treating reactions or allergies to molds is emitted in the following order 4442, 2411, 1833, 1823, 1333, 1155, 1130, 1016, 942, 933, 886, 880, 886, 784, 774, 766, 745, 743, 728, 623, 623, 594, 592, 565, 555, 524, 512, 464, 414, and 374 Hz.

Those of skill in the art will appreciate that the order in which the frequencies are emitted in the afore listed combinations can be changed without deviating from the intended purpose or altering the expected outcome.

As stated previously, a frequency may be repeated within a combination, but that it is preferred that no frequency is repeated multiple times contiguously. Similarly, it is preferred that when using an embodiment of the invention that no single combination be repeatedly used continuously without interruption. Rather, it is expected that the invention is more effective if either a combination is played once followed by an interlude, or if multiple combinations are played, then they are not played repeatedly without one or more intervening interludes.

It will be appreciated that many other different combinations of acoustic frequencies can be used in the invention. It is expected that any of the published frequencies that have been identified as potentially therapeutic may be combined together and used in the invention. Lists of such frequencies are readily available. For example, The Frequencies of Rifing—From the first frequencies discovered by Royal Rife to today: Guide to selection and use of Spooky2 frequencies, 3^{rd} Ed., 2018, by Ing. Marcello Allegretti, includes numerous tables of such frequencies and their associated uses. Those of skill in the art will be familiar with other sources of lists of frequencies that are readily available either in hard copy or electronic format.

Devices of the invention are capable of generating the acoustic frequencies of the combinations. The range of frequencies that can be used in the invention include from 1-20,000 Hz. Preferred frequencies are those that have been either demonstrated or antidotally implicated as having a therapeutic effect either on a person or non-human animal.

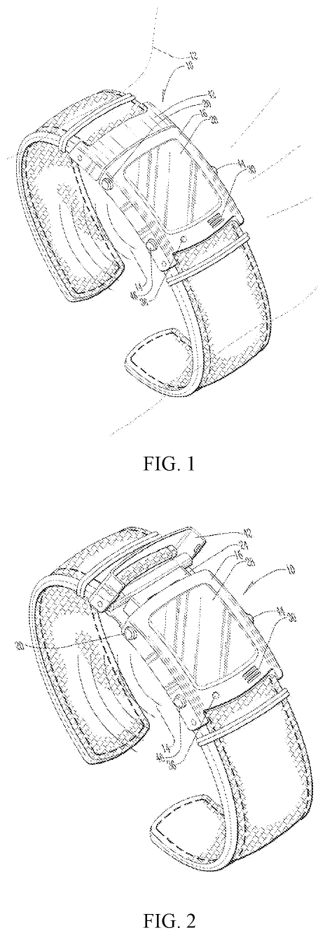

Because little energy is required to generate the acoustic frequencies, devices of the invention have low energy requirements and can be made into a variety of shapes and sizes. Devices of the invention can be worn on a wrist, leg, neck, or head. For example, a device of the invention may be incorporated into a watch, bracelet, necklace, pendant 56, belt, or other object that can be worn. See FIGS. 1 and 9. Alternatively, but less preferably, devices may be adjacent to the subject such that the subject can touch the device or receive the oscillations through another object that can is in contact with the device and through which the oscillations are transmitted to the user. For example, a device of the invention may be attached to a metal cart that the user grasps by a handle through which oscillations can be received.

While several configurations for devices of the invention exist, all of these devices require that the user's skin either be in contact with or sufficiently adjacent to the device such that the acoustic frequencies generated by the device can penetrate the user's skin and generate oscillations in at least one cell within the user such that the oscillations can be transmitted to a target within the user's body. Further, those of skill in the art will appreciate that many computer programs are available that do not require inventive skill to generate instructions to generate a series of frequencies that can be uploaded into a device.

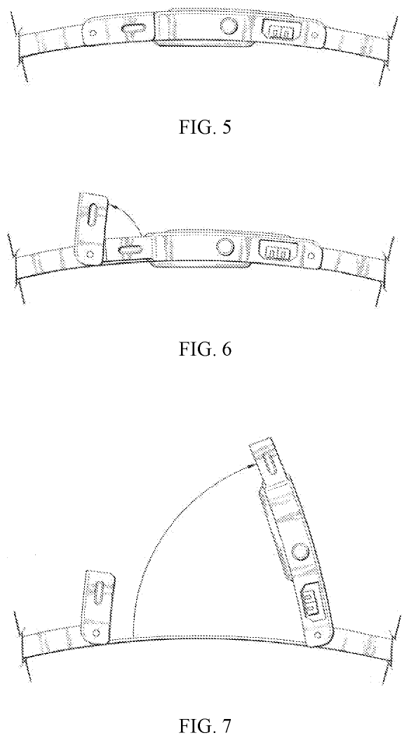

Herein, a preferred embodiment of a device of the invention, i.e. a wristwatch, is shown in FIGS. 1-8. It will be appreciated that different types of wristwatches exist and can be used in the invention.

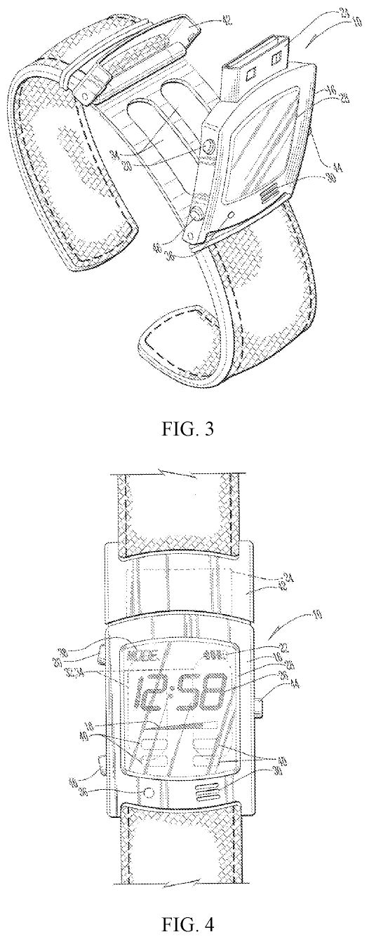

As illustrated in FIG. 1, a device of the invention can be worn a wrist 12 and appear to be only a watch 10 having a housing face 28, a housing 16, a time control (button) 44 and a backlight switch (button) 46. In addition, a watch includes a mode switch (button) 20 by which to access one or more programs or recordings of combinations of frequencies of the invention. Preferred devices also include a microphone 36 so that a user can record their voice or other acoustic signals and play them back as desired. In addition, a watch 10 will include a speaker 30 and means of protecting internal parts of the invention such as a USB pivot guard 42. In the exemplary watch, both the USB pivot guard 42 and housing 16 can be moved for access and easier updating of the programming of frequency combinations through a USB port 24. See FIGS. 2, 3 and 5-7. On the underside, or surface adjacent to the skin, of devices of the invention, one or more emitter openings 34 through which the speaker 30 can transmit frequency signals 14 are present. See FIGS. 1 and 3.

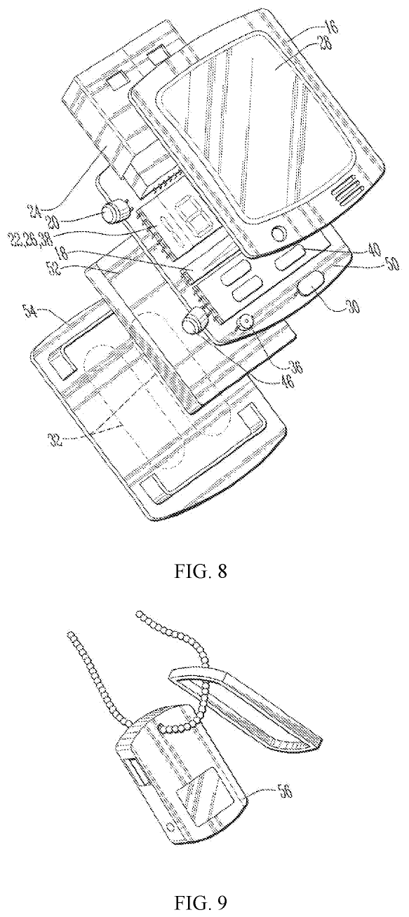

Preferably, when a device of the invention is a watch it includes a battery level indicator 22, a time display 26, one or more mode indicators 38, 40, and a volume indicator 18 and a means of adjusting the volume. The number and types of mode indicators present in part depends upon the overall design of the watch and its other features that may be present. See FIG. 4. Devices of the invention also include one or more frequency emitters 32, which may comprise a variety of shapes and configurations depending upon the overall shape and design of a device, that transmit frequencies through the emitter openings 34. In an exploded view of the exemplary watch, the internal circuit board 50, battery 52, and frequency emitter platform 54 are illustrated. See FIG. 8. Those of skill in the art that many suitable configurations for circuit boards and batteries exist and may be used in devices of the invention.

Unless defined otherwise, all technical and scientific terms used herein have the same meaning as is commonly understood by one of skill in the art to which this invention belongs at the time of filing. Terminology used herein is for the purpose of describing particular embodiments of the invention and is not intended to be limiting. The meaning and scope of terms should be clear; however, in the event of any latent ambiguity, definitions provided herein take precedent over any dictionary or extrinsic definition. Further, unless otherwise required by context, singular terms shall include pluralities and plural terms shall include the singular unless the content clearly dictates otherwise. Herein, the use of “or” means “and/or” unless stated otherwise. Furthermore, the use of the term “including”, as well as other forms such as “includes” and “included” is not limiting. As used herein, “each” refers to each member of a set or each member of a subset of a set. All patents and publications referred to herein are incorporated by reference herein.

It should be appreciated by those of skill in the art that the techniques and examples disclosed herein represent techniques discovered by the inventor to function well in the practice of the invention, and thus can be considered to constitute preferred modes of practice. However, those of skill in the art should, in light of the present disclosure, appreciate that many changes can be made in the specific embodiments which are disclosed and still obtain a like or similar result without departing from the spirit and scope of the invention. Thus, the present invention should not be limited to the exemplary embodiments and techniques illustrated herein.

What is claimed is:

1. A method of treating a user comprising:
a) positioning a device adjacent to an outer skin surface of the user; and
b) emitting a program, wherein the program comprises at least one oscillation, from the device near an outer skin surface of the user such that at least one cell within the user receives the oscillation.

2. The method of claim 1, wherein the program comprises two or more oscillations.

3. The method of claim 2, wherein each oscillation comprises a combination of multiple acoustical frequencies that are emitted together for a pre-determined amount of time.

4. The method of claim 3, wherein the acoustical frequencies are within the range of 1 to 20,000 Hz.

5. The method of claim 3, wherein the acoustical frequencies are emitted for several seconds to several minutes.

6. The method of claim 2, wherein the two or more oscillations are arranged in sequential order within the program.

7. The method of claim 1, wherein the cell is in the user's vagus nerve.

8. The method of claim 1, wherein the user is treated for pain, inflammation, acne, allergies, arthritis, ankylosing spondylitis, asthma, bursitis, neuropathy, carpel tunnel syndrome, colitis, constipation, infection, diarrhea, dizziness, Meniere's disease, tinnitus, dystonia, fibromyalgia, kidney calculi, cholecystitis, headaches, migraines, hemorrhoids, hernias, irritable bowel syndromes, laryngitis, lumbago, cramps, numbness, tremors, peripheral nervous system disease, pulmonary fibrosis, Raynaud's disease, sinusitis, Tourette's syndrome, trigeminal neuralgia, urinary tract infections, bladder disease or disorders, varicose veins and venous insufficiency, breast diseases, alopecia, amenorrhea, cysts, mastitis, ademona, dysmenorrhea, endometriosis, respiratory disease or infection, or a stress or psychological disorder.

9. The method of claim 1, wherein the emitting of the oscillation near an outer skin surface stimulates a vibrational energy to the outer skin surface and area beneath the outer skin surface.

10. The method of claim 1, wherein the device is wearable and configured to be worn around the user's wrist, ankle, neck, or waist.

11. A device having
a) a means of being worn by or attached to a user such that the device is in direct contact with or adjacent to the user's skin; and
b) a means of emitting a program, wherein the program comprises at least one oscillation that is emitted towards the user's skin such that at least one cell within the user receives the oscillation, and the oscillation comprises one or more combinations of multiple acoustical frequencies that are emitted together for a pre-determined amount of time.

12. The device of claim 11, wherein the acoustical frequencies are within the range of 1 to 20,000 Hz and are emitted for several seconds to several minutes.

13. The device of claim 11, wherein the cell is in the user's vagus nerve.

14. The device of claim 11, wherein the device is configured to be worn around the user's wrist, ankle, neck, or waist.

15. A system for treating a user comprising
a) a device having a means of being worn by or attached to a user such that the device is in direct contact with or adjacent to the user's skin; and a means of emitting a program, wherein the program comprises at least one oscillation that is emitted towards the user's skin such that at least one cell within the user receives the oscillation, and the oscillation comprises one or more combinations of multiple acoustical frequencies that are emitted together for a pre-determined amount of time; and
b) using the device to treat the user for pain, inflammation, acne, allergies, arthritis, ankylosing spondylitis, asthma, bursitis, neuropathy, carpel tunnel syndrome, colitis, constipation, infection, diarrhea, dizziness, Meniere's disease, tinnitus, dystonia, fibromyalgia, kidney calculi, cholecystitis, headaches, migraines, hemorrhoids, hernias, irritable bowel syndromes, laryngitis, lumbago, cramps, numbness, tremors, peripheral nervous system disease, pulmonary fibrosis, Raynaud's disease, sinusitis, Tourette's syndrome, trigeminal neuralgia, urinary tract infections, bladder disease or disorders, varicose veins and venous insufficiency, breast diseases, alopecia, amenorrhea, cysts, mastitis, ademona, dysmenorrhea, endometriosis, respiratory disease or infection, or a stress or psychological disorder.

16. The system of claim 15, wherein the acoustical frequencies are within the range of 1 to 20,000 Hz and are emitted for several seconds to a few minutes.

17. The system of claim 15, wherein the cell is located in the user's vagus nerve.

18. The system of claim 15, wherein the emitting of the oscillation near an outer skin surface stimulates a vibrational energy to the outer skin surface and area beneath the outer skin surface.

19. The system of claim 15, wherein the device is configured to be worn around the user's wrist, ankle, neck, or waist.
